SEQUENCE LISTING (9 sequences)

SEQ ID NO: 1 - DNA, 339 bp, Artificial Sequence
  RECOMBINANT GANODERMA LUCIDIUM IMMUNOMODULATORY DNA
atgtctgatactgctttgatcttcagattggcttgggatgttaagaagttgtcttttgattacactccaaactggggtagaggtaacccaaacaacttcattgatactgttacttttcctaaggttttgactgataaggcttacacttacagagttgctgtttctggtagaaacttgggtgttaagccatcttacgctgttgaatctgatggttctcaaaaggttaacttcttggaatacaactctggttacggtattgctgatactaacactattcaagttttcgttgttgatccagatactaacaacgatttcattatcgctcaatggaactagtaa

SEQ ID NO: 2 - protein, 111 aa, Artificial Sequence
  Recombinant Ganoderma Lucidium Immunomodulatory Protein
MSDTALIFRLAWDVKKLSFDYTPNWGRGNPNNFIDTVTFPKVLTDKAYTYRVAVSGRNLGVKPSYAVESDGSQKVNFLEYNSGYGIADTNTIQVFVVDPDTNNDFIIAQWN

SEQ ID NO: 3 - protein, 7 aa, Artificial Sequence
  Portion of Beta-strand of Recombinant Ganoderma Lucidium Immunomodulatory Protein
TPNWGRG

SEQ ID NO: 4 - protein, 7 aa, Artificial Sequence
  Portion of Beta-strand of Recombinant Ganoderma Lucidium Immunomodulatory Protein
IDTVTFP

SEQ ID NO: 5 - protein, 7 aa, Artificial Sequence
  Portion of Beta-strand of Recombinant Ganoderma Lucidium Immunomodulatory Protein
YTYRVAV

SEQ ID NO: 6 - protein, 6 aa, Artificial Sequence
  Portion of Beta-strand of Recombinant Ganoderma Lucidium Immunomodulatory Protein
RNLGVK

SEQ ID NO: 7 - protein, 5 aa, Artificial Sequence
  Portion of Beta-strand of Recombinant Ganoderma Lucidium Immunomodulatory Protein
SQKVN

SEQ ID NO: 8 - protein, 10 aa, Artificial Sequence
  Portion of Beta-strand of Recombinant Ganoderma Lucidium Immunomodulatory Protein
TIQVFVVDPD

SEQ ID NO: 9 - protein, 9 aa, Artificial Sequence
  Portion of Beta-strand of Recombinant Ganoderma Lucidium Immunomodulatory Protein
NNDFIIAQW

(19) United States
(12) Patent Application Publication
(10) Pub. No.: US 2015/0023994 A1
(43) Pub. Date: Jan. 22, 2015

(54) RECOMBINANT GANODERMA LUCIDIUM IMMUNOMODULATORY PROTEIN (rLZ-8) AND USES THEREOF

(71) Applicants: Fei SUN, Shanghai (CN); Chongyang LIANG, Shanghai (CN); Xitian ZHANG, Shanghai (CN)

(72) Inventors: Fei SUN, Shanghai (CN); Chongyang LIANG, Shanghai (CN); Xitian ZHANG, Shanghai (CN)

(21) Appl. No.: 14/511,781

(22) Filed: Oct. 10, 2014

Related U.S. Application Data
(62) Division of application No. 12/829,653, filed on Jul. 2, 2010, now abandoned.
Application No. 12/829,653 is a continuation of application No. PCT/CN2008/002142, filed on Dec. 30, 2008.

(30) Foreign Application Priority Data
Jan. 3, 2008 (CN) 200810050206.X

(51) Int. Cl. C07K 14/37

(52) U.S. Cl. CPC C07K 14/37
USPC 424/185.1

(57) ABSTRACT

Provided are the use of the recombinant Ganoderma lucidum immunomodulatory protein (rLZ-8) for the manufacturing of a medicament for antitumor, increasing leukocyte and inhibiting immunological rejection and the pharmaceutical composition comprising the rLZ-8 protein, wherein the rLZ-8 protein is expressed from pichia yeast.

### FIELD OF TECHNOLOGY

The invention falls into the field of biomedical engineering, involving medical uses of recombinant Ganoderma Lucidum Immunoregulatory Protein for anti-tumor, raising leucocyte and immune suppression.

### BACKGROUND TECHNOLOGY

In existent researching files, a specified kind of proteins come from Ganoderma Lucidum exert extensive immune regulatory activities including regulation, red cells agglutinate, gene modulation of adhesion molecule, suppressing allergy reaction and immune anti-tumor effect.

The separation and purification of small-molecule protein from the extractive of Ganoderma mycelium was done by a Japanese Kino and others in 1989 (Kohsuke Kino et al., J. Bio. Chem. 1989, 1:472-478), it was named LZ-8, its amino acid sequence and physiological activity of immunity was also tested which indicates that the sequence of protein of LZ-8 is made up of 110 amino acid residues, acetylation, the molecular weight is 12.4 KD, and the isoelectric point is 4.4.

The crystal structure of LZ-8 has been published, its main features are as follows: The crystal structure of LZ-8 was solved at 2.10 Å resolution. The crystal belongs to the space group of P212121, with a unit cell of a=33.19 Å, b=86.99 Å, c=92.13 Å and α=β=γ=90°. The overall fold of LZ-8 consists of an N-terminal dimerization domain and a C-terminal FNIII domain. The N-terminal domain is composed of an α-helix and a β-strand that sustains the dimerization via domain swapping, forming a dumb-bell-shaped dimer. The C-terminal FNIII domain belongs to the immunoglobulin-like beta-sandwich fold and comprises a sandwich structure of two β-sheets (I and II) formed by β-strands A-B-E and G-F-C-D, respectively, amino acid sequence of β-strand: A. 21-TPNWGRG-27; B. 34-IDTVTFP-39; C. 48-YTYRVAV-54; D. 57-RNLGVK P-63; E. 72-SQKVN-76; F. 91-TIQVFVVDPD-100; G 102-NNDFHAQW-110.

Recombinant human Granulocyte colony-stimulating factor (G-CSF) is the only clinical gene engineering preparation used with certain cancer patients to accelerate recovery from neutropenia after chemotherapy, allowing higher-intensity treatment regimens.

Although years of practice using in clinic have proved the effect of G-CSF, its all brands of production are marked with adverse reactions like “The common side effect you/your child may experience is aching in the bones and muscles, fever, skin eruption, nausea. Serious allergic reactions can also happen, these reactions can cause a rash over the whole body, shortness of breath, wheezing, a drop in blood pressure, swelling around the mouth or eyes, fast pulse, or swearing.”

The invention results show that recombinant LZ-8 was extremely effective in the treatment and prevention of Leucopenia having various causes, and it is better than all existing clinical drugs in the respects of dosage, effect and clinical adverse reactions. Animal experiments gave results that side effect of recombinant LZ-8 was highly lower than that of G-CSF.

The previous reports about LZ-8 suggested that it kill tumor cells through immune regulatory pathways. In this invention, inventor found that rLZ-8 could kill HL-60, NB4, K562 tumor cells directly rather than indirect immune regulatory pathways.

The major function of Ganoderma Lucidum Immune Regulatory Protein lies in that it stimulates the hyperplasia of peripheral lymphocytes and spleen cells, induces the macrophage both in human and animals to secrete various cell factors (as in interleukin, tumor necrosis factor and interferon, etc.), and defending and dispelling the infringement of the causative agent, safeguards and maintains the health and to achieve the immune regulatory function. This invention suggest that rlZ-8 could prevent systemic allergic reactions and immune rejection after organ transplantation effectively.

In conclusion, inventor has finish the works about rLZ-8 as below: Offering a efficient-level expression of rLZ-8 engineering bacteria; Providing a 100 L scale of preparation method of technique and purification technology to improve the yield and overcome the problems of existing small-scale production and low efficiency; Presenting 1.8 Å X-ray structure of LZ-8, determined by single anomalous diffraction (SAD) using the anomalous signal of bromide ions present in the crystal for phasing; Making the anti-tumor effect of rLZ-8 and its function of elevating the leukocytes public; The results of Flow Cytometry assay showed that rLZ-8 has a strong lethal effect on leukemia cells NB4, K562 and HL-60, detection of apoptosis flow cytometry further proved rLZ-8 could induce leukemia cells apoptosis in vitro; Hemolysis experiment, rat bone marrow experiment and erythropoietin cohesion experiment all showed that no influence on normal cells is confirmed; Experiments on tumor-bearing mice showed that rLZ-8 could inhibit Ehrlich ascites tumor in mice S180 and H22 implanted hepatoma cell growth in vivo; Fluorescent labeling assay showed that rLZ-8 may induce the apoptosis destruction of the specific binding of tumor cells and killing tumor cells.

### THE CONTENT OF INVENTION

The invention falls into the field of biomedical engineering, involving medical uses of recombinant Ganoderma Lucidum Immunoregulatory Protein for anti-tumor, raising leucocyte and immune suppression. In the invention, Owing to the genetic code preference of Pichia pastoris, re-designs LZ-8 full-gene synthesis encoding sequence based on the original Ganoderma Lucidum Immune immunomodulatory protein gene sequence, recombinant express LZ-8 in Pichia pastoris.

To elucidate the structural basis of this uniqueness, crystal structure of LZ-8 was solved by X-ray diffraction. The crystal was grown at 289K using the hanging drop vapor diffusion method, culture condition is 1.75 M ammonium sulfate, 0.1 M Tris-HCl, pH 6.0 and 6.4% polyethylene glycol 400. rLZ-8's X-ray diffraction data was collected at 1.80 Å resolution, and Data were processed with the Program MOSFLM11 and scaled with SCALA12 and CCP4. domain. The N-terminal domain is composed of an α-helix and a β-strand that sustains the dimerization via domain swapping, forming a dumb-bell-shaped dimer. The C-terminal FNIII domain belongs to the immunoglobulin-like beta-sandwich fold and comprises a sandwich structure of two β-sheets (I and II).

In vitro anti-tumor assay, mix K562 and NB4 cells into 24-orifice plate respectively, and supplied rLZ-8 in different concentrations, a significant direct cytotoxic effect of rLZ-8 was examined with MTT and observed under microscope. In vivo tumor inhibition assay, rLZ-8 treatment groups were given doses of tail venous injection respectively, in 10 consecutive days. Draw blood from orbital venous plexus 10 days before and after medication respectively, test the number of white blood cell (WBC). Killed all mice the next day of drug withdrawal at cervical dislocation, dissect anatomically and took out the tumors, weigh tumors, calculated the inhibition rate. Conclusion: Experimental dose of rLZ-8 has a very effective anti-tumor activity in vivo and in vitro.

In the invention, Fluorchrome (Fluorescein-5-Isothiocyanate, FITC) was used to label rLZ-8 , formed FITC-rLZ-8, hatched it with H160 cells, rat myocardial tissues and rabbit chondrocytes, collected and washed cells, under fluorescence microscope observation, with FITC-rLZ-8 incubation of 1 h and 6 h the HL-60 cells, with strong green fluorescence, compared with rat myocardial tissues and rabbit chondrocytes, there is a significant difference. These results served to show that a certain receptor could be recognized by rLZ-8. It is inferred that the recognition mechanism may be related to Oligosaccharide Link on the surface of HL-60 cells, because there were two carbohydrate chain binding sites on C-terminal FNIII domain of rLZ-8, and any Oligosaccharide Links have never been found on normal cells' surface. Base on this, the invention suggested that specific killing effect of rLZ-8 could be due to an association with recognizing the receptors on the surface of cells.

For further studying the anti-tumor mechanism, the apoptotic percentage of rLZ-8-treated K562 and H160 cells was measured by flow cytometry using propidium iodode (PI) stain and ANNEXIN V&FITC stain, the results drew the conclusion that apoptosis of cells is one of the ways for rLZ-8 to kill tumor cells.

The invention disclosed rLZ-8 has some effects in the prevention and treatment of leucopenia. In “rLZ-8 effect on low-interleukin rat model” assay, After successful modeling, they were given to the above groups respective dose of rLZ-8 and positive drugs (Genlei™ Scimax™) treatment, normal control group and CP groups were given same normal saline, on the 3rd , 7th and 14th day of the treatment to drew venous blood from rats tail respectively, tested the numbers of WBC. Compared WBC changes before and after the treatment for analysis of drug efficacy. Compared with the CP group, treatment on the 3rd day of rLZ-8 drug group in rats had significantly higher WBC, the difference is very significant, it became normal on the 7th day of treatment.

In the treatment of mice model of leucopenia established by radiation, the mice were irradiated by 7.5 Gy γ-ray (180 mV, 15 mA) and then divided into seven groups (n=10). Together with modeling, mice in each group were administered with Genlei™ Scimax™ and rLZ-8 for 9 days, drew blood from tail at 5^{th}, 7^{th}, 9^{th} day, tested the number of WBC, weight spleen, calculated index of spleen. In the prevention assay, the same method was used, and each group except control ones were administered in advance. Both of results above indicated rLZ-8 could prevent and treat Leucopenia caused by radiation.

The invention proved that rLZ-8 could not produce hemolysis, coagulation and abnormal influence on rat myelogram. The invention described that rLZ-8 could be used as an immunosuppressive agent to treat Systemic Allergic Reactions in rats stimulated by BSA. The invention included that rLZ-8 core components of pharmaceutical preparations contained Recombinant Ganoderma Lucidum Immune Regulatory Protein and acceptable optional pharmaceutical adjuvant. rLZ-8 pharmaceutical preparations can be oral delivery and non-intestinal drug delivery.

### DESCRIPTION TO MANUAL FIGURES

FIG. 1. rLZ-8 crystal structure captions

FIG. 2. rLZ-8 on NB4 tumor cells in vitro results wherein A is a normal control group; B is a positive control group; and C-J are rLZ-8 groups follow the order 0.78 μg·ml⁻¹, 1.56 μg·ml⁻¹, 3.125 μg·ml⁻¹, 6.25 μg·ml⁻¹, 12.5 μg·ml⁻¹, 25 μg·ml⁻¹, 50 μg·ml⁻¹, 100 μg·ml⁻¹

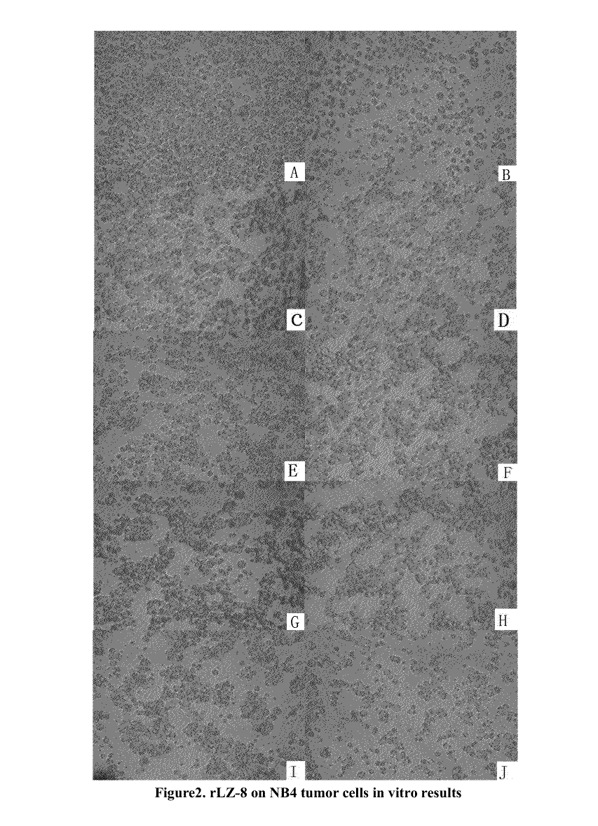

FIG. 3. rLZ-8 on K562 tumor cells in vitro results wherein A is a normal control group; B is a positive drug control group; and C-H are rLZ-8 groups follow the order 3.125 μg·ml⁻¹, 6.25 μg·ml⁻¹, 12.5 μg·ml⁻¹, 25 μg·ml⁻¹, 50 μg·ml⁻¹, 100 μg·ml⁻¹

FIGS. 4A and 4B. rLZ-8-induced apoptosis of K562 and NB4 cells, PI single staining test results wherein C1 is a K562 normal control group; I1 is rLZ-8 group (K562, 0.1 μg/ml; I2 is rLZ-8 group (K562, 0.5 μg/ml; and I3 is rLZ-8 group (K562, 2.5 μg/ml) and wherein C2 is NB4 Normal control group; H1 is rLZ-8 group (NB4, 0.1 μg/ml; H2 is rLZ-8 group (NB4, 0.5 μg/ml) : and H3 is rLZ-8 group (NB4, 2.5 μg/ml); FIG. 4B.

FIGS. 5A and 5B. rLZ-8-induced apoptosis of H160 and NB4 cells, Annexin V/PI double staining test results wherein C1 is NB4 Normal control group; P1 is a positive drug control group; I1 is rLZ-8 group (NB4, 0.1 μg/ml); I2 is rLZ-8 group (NB4, 0.5 μg/ml); I3 is rLZ-8 group (NB4, 2.5 μg/ml) and wherein C2 is NB4 Normal control group; P2 is a positive drug control group; H1 is rLZ-8 group (HL-60, 0.1 μg/ml); H2 is rLZ-8 group (HL-60, 0.5 μg/ml); and H3 is rLZ-8 group (HL-60, 2.5 μg/ml)

FIG. 6 Inoculated S180 Ehrlich ascites tumor cells in mice body weight change

FIG. 7 Inoculated H22 tumor cells implanted in mice body weight change

FIG. 8. FITC-rLZ-8 (100 ng·ml⁻¹) rat myocardial tissue markers (dark, DIC field)

FIG. 9 FITC-rLZ-8 (100 ng·ml⁻¹) rabbit chondrocyte markers (dark, DIC field)

FIG. 10 FITC-rLZ-8 (100 ng·ml⁻¹) HL-60 cell markers (dark, DIC field)

FIG. 11 Rat myelogram influenced by rLZ-8 wherein A is a normal control group; B is rLZ-8 (15 mg·kg⁻¹); C is rLZ-8 (30 mg·kg⁻¹); and D is rLZ-8 (60 mg·kg⁻¹)

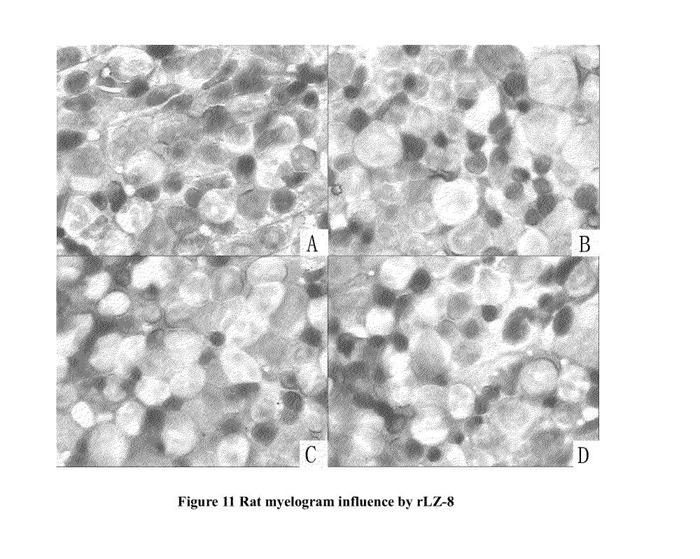

### EXAMPLES

### Example 1

### Obtaining the Recombinant Ganoderma Lucidum Immune Regulatory Protein

### 1.1 Synthetic rLZ-8 Genes, Construction of Engineering Bacteria, Construction and Screening

Owing to the genetic code preference of Pichia pastoris, re-designed LZ-8 full-gene synthesis encoding sequence (SEQ ID 1) based on the original Ganoderma Lucidum Immune immunomodulatory protein gene sequence, and linked with yeast α-factor leader peptide coding sequence to become a fusion gene, cloned α-LZ-8 gene into a pMD18-T carrier, linearized the carriers of correct sequencing and implanted into yeast gene genome, and screening methanol by using highly efficient Mut⁺ strains on the MM and MD plates.

### 1.2 The Expression of rLZ-8 Engineering Bacteria

Expression of the fermentation scale, temperature, speed of rotation, pH values, liquid volume, supplement of methanol and other detections have established the optimized process of yeast in the 100 L fermentor scale expression rLZ-8 conditions. We have designed the poilt fermentation medium formula in accordance with the physical and chemical properties of rLZ-8. The rLZ-8 output is about 800 mg·L⁻¹.

### 1.3 Purification Process of rLZ-8

Fermentation broth centrifugal separator→supernatant tubular separator→ultra filtration→cation exchange column purification→AKTA protein purification workstation for preparation of the target protein→strong anion-exchange chromatography purification→hydrophobic interaction column→gel filtration chromatography.

### 1.4 RLZ-8 Purity and Molecular Weight Determination

Purity analysis of separation and purification using RP-HPLC, the purity of rLZ-8 is >99%. Evaluation of laser-flight mass spectrometry of Recombinant expression of rLZ-8 molecular weight is 12,722 Da.

### 1.5 Determination of rLZ-8 Higher Spatial Structure

We obtained the single-crystal of 0.2 cm×0.2 cm×0.2 cm by using suspension vapor diffusion method. Selenium crystals MacChessF2 beamline have collected 1.8 angstroms (A) resolution crystal diffraction data. Non-selenium mother crystal collected 1.8 angstroms resolution crystal diffraction data on MarResearch 345dtd image plain diffraction data collection system. The monomer structure of subunit of Recombinant Ganoderma Lucidum Immune Regulatory Protein rLZ-8 (SEQ ID 2) is composed by α-helix, β-strand, 2βsheets formed by β-strands A-B-E and G-F-C-D, respectively, amino acid sequence of β-strand: A. 21-TPNWGRG-27 (SEQ ID 3); B. 34-IDTVTFP-39 (SEQ ID 4); C. 48-YTYRVAV-54 (SEQ ID 5); D. 57-RNLGVK P-63 (SEQ ID 6); E. 72-SQKVN-76 (SEQ ID 7); F. 91-TIQVFVVDPD-100 (SEQ ID 8); G. 102-NNDFIIAQW-110 (SEQ ID 9).

### Example 2

### Killing Effect of rLZ-8 on Human Promyelocytic Leukemia NB4 Cells

After filtration and degerming, we prepared 8 concentrations using IMDM culture medium which are respectively 0.78 μg·ml⁻¹, 1.56 μg·ml⁻¹, 3.125 μg·ml⁻¹, 6.25 μg·ml⁻¹, 12.5 μg·ml⁻¹, 25 μg·ml⁻¹, 50 μg·ml⁻¹, 100 μg·ml⁻¹.

On 96-orifice culture medium plate, the pilot orifice plus NB4 tumor cells 0.1 ml and rLZ-8 0.1 ml, rLZ-8 concentrates from low to high; negative control group plus NB4 tumor cells and culture medium of 0.1 ml respectively; positive drug control group arsenic trioxide As₂O₃; make six-aperture multipunches for each group. Placing in 37° C., 5% CO₂ incubator 48 h, adding MTT 15 μl (5 mg ml⁻¹) prior to the termination of 4 h, after the termination of cell culture by adding 100 μl 0.1 mol L⁻¹ hydrochloric acid isopropyl alcohol, testing on the enzyme-linked immunosorbent OD_{570 nm} values.

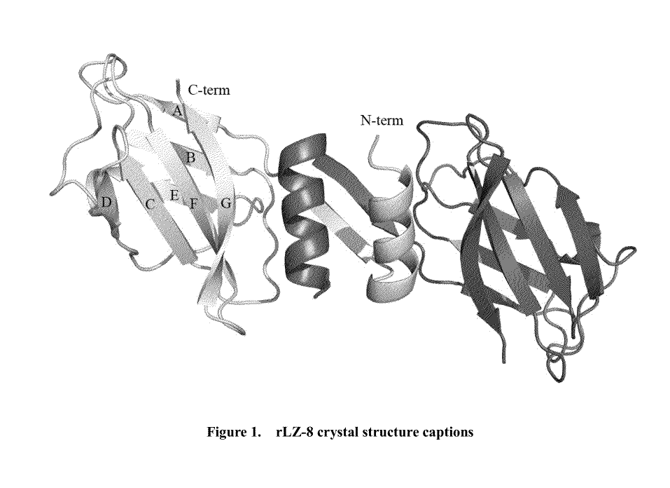

Table 1 and FIG. 1 showed that rLZ-8 drug group at OD_{570 nm} optical absorption value and NB4 normal control group, there are significant differences that rLZ-8 has a strong lethal effect in vitro on NB4 tumor cells.

TABLE 1
rLZ-8's lethal effect in vitro on NB4 tumor cells ( x̅ ± s, n = 6)

| | Dosage | | Growth inhibition |
|---|---|---|---|
| Groups | (μg ml⁻¹) | OD_{570 nm} | rate (%) |
| Normal control | — | 1.16 ± 0.020 | — |
| group | | | |
| Positive drug | 10 | 0.33 ± 0.01* | 72 |
| control group | | | |
| rLZ-8 groups | 0.78 | 0.71 ± 0.03* | 39 |
| | 1.56 | 0.65 ± 0.05* | 44 |
| | 3.125 | 0.53 ± 0.04* | 54 |
| | 6.25 | 0.45 ± 0.02* | 61 |
| | 12.5 | 0.30 ± 0.04* | 74 |
| | 25 | 0.22 ± 0.01* | 81 |
| | 50 | 0.11 ± 0.01* | 91 |
| | 100 | 0.08 ± 0.01* | 93 |

Comparison of drug group with NB4 normal control group,
*p < 0.01

### Example 3

### Lethal Effect of rLZ-8 on Human Chronic Myelogenous Leukemia K562 Cells

After sterilization of rLZ-8, we prepared 6 concentrations using IMDM culture medium, which are respectively 3.125 μg·ml⁻¹, 6.25 μg·ml⁻¹, 12.5 μg·ml⁻¹, 25 μg·ml⁻¹, 50 μg·ml⁻¹, 100 μg·ml⁻¹.

On 96-orifice culture plate, the pilot aperture plus K562 tumor cells 0.1 ml and rLZ-8 0.1 ml, rLZ-8 concentrations from low to high; negative control group plus K562 tumor cells and culture medium of the 0.1 ml respectively; positive drug control group arsenic trioxide As₂O₃; make six-aperture multipunches for each group. Placing in 37° C., 5% CO₂ incubator 48 h, adding MTT 15 μl (5 mg ml⁻¹) prior to the termination of 4 h, after the termination of cell culture by adding 100 μl 0.1 mol L⁻¹ hydrochloric acid isopropyl alcohol, testing OD values _{570 nm} on the enzyme immunosorbent detector.

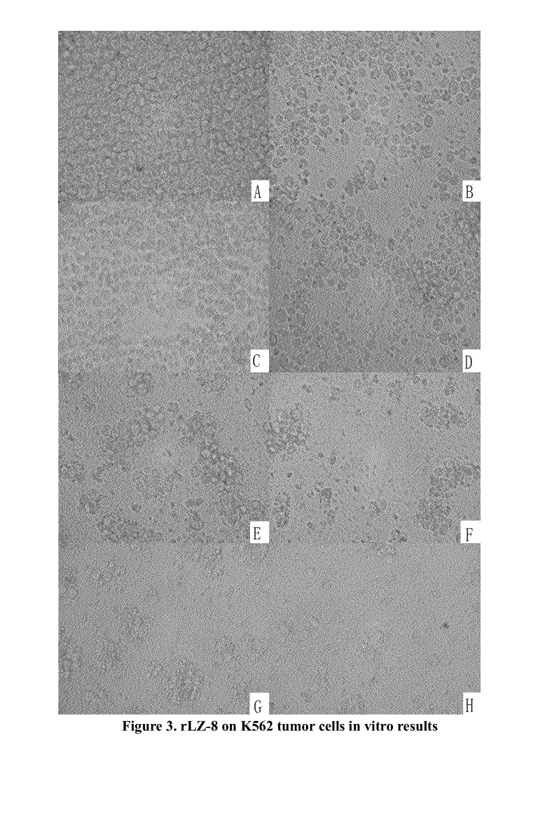

Table 2 and FIG. 3 show that rLZ-8 drug group at OD_{570 nm} optical absorption value and K562 normal control group, there are significant differences that rLZ-8 has a strong lethal effect in vitro on K562 tumor cells.

TABLE 2
rLZ-8 lethal effect on tumor cells K562 in vitro ( x̅ ± s, n = 6)

| | Dosage | | Growth inhibition |
|---|---|---|---|
| Groups | (μg ml⁻¹) | OD_{570 nm} | rate (%) |
| Normal control | — | 1.01 ± 0.01 | — |
| group | | | |
| Positive drug | 10 | 0.22 ± 0.03* | 78.2 |
| control group | | | |
| rLZ-8 groups | 3.125 | 0.66 ± 0.03* | 34.7 |
| | 6.25 | 0.58 ± 0.03* | 42.6 |
| | 12.5 | 0.52 ± 0.05* | 48.5 |
| | 25 | 0.31 ± 0.02* | 69.3 |
| | 50 | 0.25 ± 0.04* | 75.2 |
| | 100 | 0.19 ± 0.03* | 81.2 |

Comparison of drug groups with K562 normal control group,
*p < 0.01

### Example 4

### rLZ-8 Influence on Apoptosis of Blood Tumor Cell

### 1. PI Single Stained Flow Cytometry

Fluorescence microscope, model Leica ASLMD, K562, NB4. rLZ-8 is classified into high, medium and low three-dose groups respectively, now we prepare them with IMDM culture medium containing 2% FCS into the preparation of 2.5 μg·ml⁻¹, 0.5 μg/ml, 0.1 μg·ml⁻¹. Propidium bromide (PI) 50 μg·ml⁻¹.

Establish K562 as the normal control group, rLZ-8 low-dose group (0.1 μg·ml⁻¹), rLZ-8 medium dose group (0.5 μg·ml⁻¹), rLZ-8 high-dose group, (2.5 μg·ml⁻¹); set NB4 as the normal control group, rLZ-8 low-dose group (0.1 μg·ml⁻¹), rLZ-8 medium dose group (0.5 μg·ml⁻¹), rLZ-8 high-dose group, (2.5 μg·ml⁻¹).

Mix K562 and NB4 cells into 24-orifice plate respectively, and supply rLZ-8 in different concentrates, 1 ml/orifice, set 3-orifice multipunches for each group. Put them in 37° C., 5% CO₂ incubator for 24 h, collect the cells of each concentrate, PBS washes twice and regulates the cell density to 1×10⁶/ml, 70% ice ethanol fixation, adjusts to −20° C. and stays overnight. Wash the cells with PBS twice after fixing the cells, add PI (end density 50 μg·ml⁻¹) for room temperature and lucifuged hatching 10 min, 1000 r·min⁻¹ centrifuge 5 min, clear supernatant with disposable 400 μl PBS re-suspension precipitation. And test on computer in 1 h.

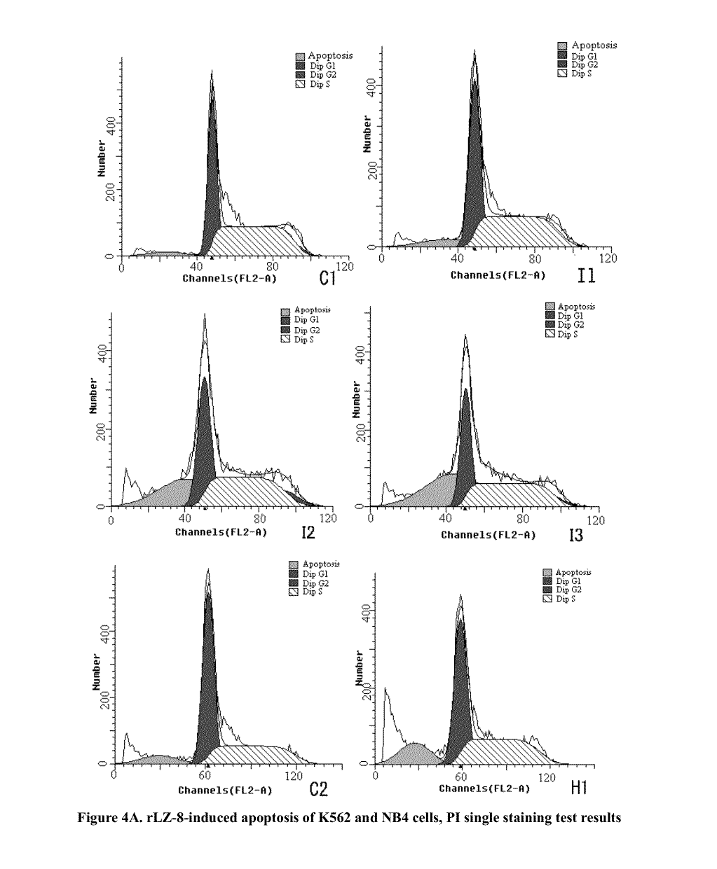

Table 3 and FIGS. 4A and 4B show that, comparison with K562 and NB4 normal control group, rLZ-8 drug group apoptosis rates increases, we may draw the conclusion that apoptosis of cells is one of the ways for rLZ-8 to kill tumor cells.

TABLE 3
rLZ-8 induced apoptosis rate on K562 and NB4 (%)

| | Apoptosis rate | | | |
|---|---|---|---|---|
| | Normal | | | |
| | control | rLZ-8 | rLZ-8 | rLZ-8 |
| | group | (0.1 μg · ml⁻¹) | (0.5 μg · ml⁻¹) | (2.5 μg · ml⁻¹) |
| K562 | 8.89 | 16.43 | 17.91 | 21.57 |
| NB4 | 4.06 | 9.23 | 27.51 | 38.60 |

### 2. Annexin V-FITC Flow Cytometry Kit Tests Apoptosis of Cells

FACS Calibur flow cytometry, U.S. Becton-Dickinson.Company. NB4, HL-60. rLZ-8 is prepared with IMDM culture medium containing 2% FCS into the preparation of 0.1 μg·ml⁻¹, 0.5 μg·ml⁻¹, 2.5 μg·ml⁻¹, arsenic trioxide (As₂O₃) 0.5 μg·ml⁻¹ Annexin V-FITC kit: combining buffer solution 4×; Propidium iodide solution (PI), 20 μg·ml⁻¹, 0.2 ml; recombinant human Annexin V/FITC, 0.1 ml.

Establish NB4 normal control group, positive drug group (As₂O₃ 0.5 μg·ml⁻¹), protein low-dose group (0.1 μg·ml⁻¹), medium-dose group (0.5 μg·ml⁻¹), high-dose group, (2.5 μg·ml⁻¹); establish HL-60 normal control group, protein low-dose group (0.1 μg·ml⁻¹), medium-dose group (0.5 μg·ml⁻¹), high-dose group, (2.5 μg·ml⁻¹).

Mix NB4 and HL-60 cell into 24-orifice plate respectively, 1 ml/hole supply rLZ-8 in different concentrates, set 3-orifice multipunches for each group. Put them in 37° C., 5% CO₂ incubator for 24 h, collect the cells of each concentrate, washes the cells twice with pre-cooled PBS 4° C., with 250 μl binding buffer solution re-suspending cells and regulates the cell density to 1×10⁶/ml, taking out 100 μl to 5 ml streaming tube, and add 5 μl Annexin V/FITC and 10 μl 20 μg·ml⁻¹PI solution, after mixing put it in room temperature and lucifuged hatching 15 min, add 400 μl PBS by flow cytometry analysis.

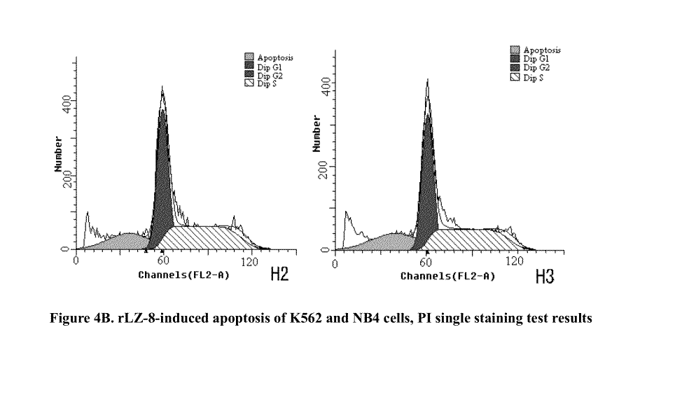

FIGS. 5A and 5B and Table 4 show that NB4-rLZ-8 group and HL-60-rLZ-8 group, the apoptosis rate is significantly higher than the normal control group, and, the apoptosis of HL-60-rLZ-8 group also rises with rLZ-8 concentration increase.

TABLE 4
rLZ-8 inducible apoptosis rate on NB4 and HL-60 cell (%)

| | Apoptosis rate | | | | |
|---|---|---|---|---|---|
| | Normal control | Positive drug | rLZ-8 | rLZ-8 | rLZ-8 |
| | group | group | (0.1 μg · ml⁻¹) | (0.5 μg · ml⁻¹) | (2.5 μg · ml⁻¹) |
| NB4 | 6.1 | 23.4 | 21.0 | 22.3 | 34.0 |
| HL-60 | 0.4 | 39.7 | 30.5 | 40.7 | 47.7 |

### Example 5

### rLZ-8 on Mouse S180 Ehrlich Ascites Tumor Inhibition

Mice, 18-22 g, males and females were equally divided, provided by Jilin University Laboratory Animal Center; Mice Ehrlich ascites cells strain, provided by our laboratory S180; Cyclophosphamide (CTX), offered by Jiangsu Hengrui Medicine Co., Ltd., lot number: 06101921. S180 ascites tumors and solid tumors in experiment group were divided into normal control group, negative control group, positive control group, rLZ-8 low-dose treatment group (0.25 mg·kg⁻¹), rLZ-8 medium-dose treatment group (0.5 mg·kg⁻¹), rLZ-8 high-dose treatment group (1 mg·kg⁻¹). For each group 10 mice.

Select well-growing S180 cells, dilute appropriately with sterile saline into a tumor cell suspension, cell counts for 10⁷·L⁻¹, under the right armpit of each mouse inoculated subcutaneously 0.2 ml (except for the normal control group). Treatment after 24 h of vaccination. Normal control group and negative control group were given saline 0.2 ml·each⁻¹·d⁻¹, intraperitoneal injection; positive control group were given cyclophosphamide 20 mg·kg⁻¹, 0.2 ml·each⁻¹·d⁻¹, intraperitoneal injection. rLZ-8 treatment groups were given doses of tail venous injection respectively, 0.2 ml·each⁻¹·d⁻¹, in 10 consecutive days. Draw blood from orbital venous plexus 10 days before and after medication respectively, sent to Jilin University No. 1 Hospital clinical laboratory to test and analyze the number of WBC. Kill all mice the next day of drug withdrawal at cervical dislocation, dissect anatomically and take out the tumors, weigh tumors, calculate the inhibition rate using the following equation:

Inhibition rate (%)=(average tumore weight of the control group−average tumore weight of the experiment group)/average tumor weight of the control group×100%

Select well-growing S180 cells, dilute appropriately with sterile saline into a tumor cell suspension, cell counts for 10⁷·L⁻¹, each mouse intraperitoneal inoculation 0.2 ml (except for the normal control group). Treatment after 24 h of vaccination. Normal control group and negative control group were given saline 0.2 ml·each⁻¹·d⁻¹, intraperitoneal injection; positive control group were given cyclophosphamide 20 mg·kg⁻¹, 0.2 ml·each⁻¹·d⁻¹, intraperitoneal injection. rLZ-8 treatment group were given doses of tail venous injection respectively, 0.2 ml·each⁻¹·d⁻¹, in 10 consecutive days. Daily weighing, observe the weight changes of mice, drawing weight growth curves.

As can be seen from Table 5, three doses of rLZ-8 can inhibit the growth of S180, inhibition rates are 16.8%, 25.7% and 45.5%. There are very significant differences (p<0.01), rLZ-8 treatment group tumor weight compared with the negative control group.

As can be seen from Table 6, before medication, the WBC numbers in mice are at the same level for all the groups, compared with the negative control group showed no differences (p>0.05). 10 days after the treatment, the negative control group WBC numbers were higher than the normal control group, rLZ-8 low-dose group and medium-dose group WBC numbers and negative control group showed no differences (p>0.05), high-dose group and the normal control group also without differences (p>0.05), the positive control group WBC numbers significantly decreased, and compared with normal control group and negative control group there were great differences (p<0.01).

TABLE 5
rLZ-8 effect on mouse transplanted S180 tumor inhibition
( x̅ ± s, n = 10)

| | Dose | Tumor weight | Inhibition |
|---|---|---|---|
| Groups | (mg · kg⁻¹) | (g) | rate (%) |
| Negative control | — | 1.01 ± 0.03 | — |
| group | | | |
| CTX group | 20 | 0.35 ± 0.02* | 65.3 |
| rLZ-8 low-dose | 0.25 | 0.84 ± 0.03* | 16.8 |
| group | | | |
| rLZ-8 medium-dose | 0.5 | 0.75 ± 0.02* | 25.7 |
| group | | | |
| rLZ-8 high-dose | 1 | 0.55 ± 0.03* | 45.5 |
| group | | | |

Comparison with the negative control group,
*p < 0.01

TABLE 6
rLZ-8 effect on mouse transplanted S180 interleukin
( x̅ ± s, n = 10)

| | | Number of leukocytes | |
|---|---|---|---|
| | Dose | Before | 10^{th} day of |
| Groups | (mg · kg⁻¹) | medication | medication |
| Normal control | — | 9.49 ± 0.27 | 9.54 ± 0.33 |
| group | | | |
| Negative control | — | 9.54 ± 0.25 | 10.44 ± 0.34 |
| group | | | |
| CTX group | 20 | 9.56 ± 0.31* | 4.41 ± 0.25* |
| rLZ-8 low-dose | 0.25 | 9.48 ± 0.30* | 10.44 ± 0.18** |
| group | | | |
| rLZ-8 medium-dose | 0.5 | 9.43 ± 0.44* | 10.34 ± 0.31** |
| group | | | |
| rLZ-8 high-dose | 1 | 9.49 ± 0.36* | 9.55 ± 0.36** |
| group | | | |

Comparison with the negative control group,
*p < 0.01;
**p < 0.05

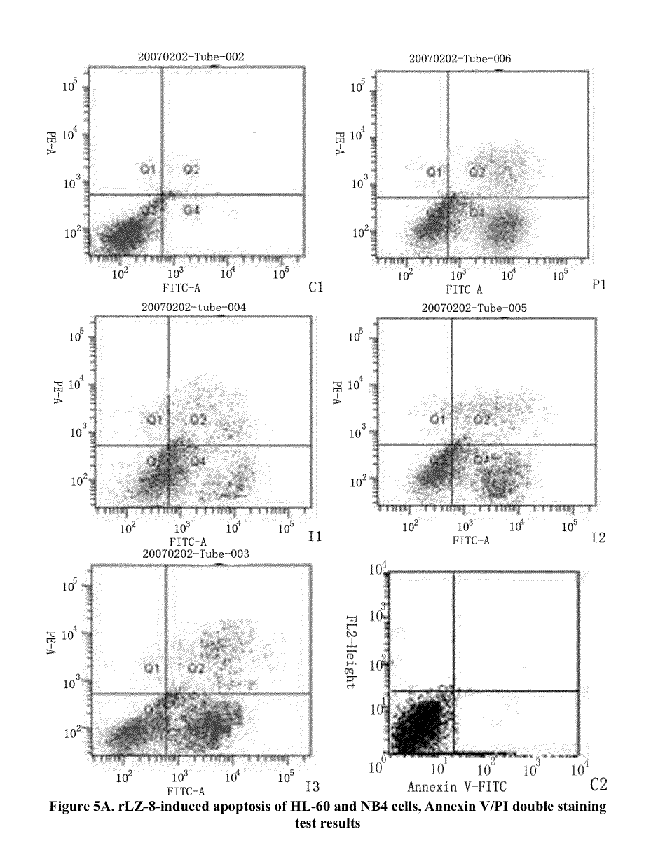

S180 ascites tumor inhibition experiment results: The experiment results showed that mice ascites in all the groups appeared basically at the same time, the negative control group of mice body weight increased rapidly, reduced survival time. From FIG. 6 we could see that mice in rLZ-8 group the average weight of growth trend than the normal group, but more than the negative control group is relatively small. Note rLZ-8 to a certain extent, inhibited mouse peritoneal S180 tumor cells growth.

### Example 6

### rLZ-8 on Mouse Hepatoma Cell H22 Inhibition Experiment

Mice, weight 18-22 g, males and females were divided equally, from Jilin University Laboratory Animal Center. Mouse hepatoma cell strain H22, offered by our laboratory. Cyclophosphamide (CTX), offered by Jiangsu Hengrui Medicine Co., Ltd., lot number: 06101921.

H22 hepatoma cells experiment group were divided into normal control group, negative control group, positive control group, rLZ-8 low-dose treatment group (0.25 mg·kg⁻¹), rLZ-8 medium-dose treatment group (0.5 mg·kg⁻¹), rLZ-8 high-dose treatment group (1 mg·kg⁻¹). For each group 10 mice.

H22 subcutaneous tumor tumor inhibition experiment methods: Select well-growing H22 cells, dilute appropriately with sterile saline into a tumor cell suspension, cell counts for 10⁷·L⁻¹, under the right armpit of each mouse inoculated subcutaneously 0.2 ml (except for the normal control group). Treatment after 24 h of vaccination. Normal control group and negative control group were given saline 0.2 ml·each⁻¹·d⁻¹, intraperitoneal injection; positive control group were given cyclophosphamide 20 mg·kg⁻¹, 0.2 ml·each⁻¹·d⁻¹, intraperitoneal injection. rLZ-8 treatment groups were given doses of tail venous injection respectively, 0.2 ml·each⁻¹·d⁻¹, in 10 consecutive days. Draw blood from orbital venous plexus 10 days before and after medication respectively, sent to Jilin University No. 1 Hospital clinical laboratory to test and analyze the number of WBC. Kill all the mice the next day of drug withdrawal at cervical dislocation, dissected anatomically and took out the tumors, weigh tumors, calculated the inhibition rate using the following equation:

Inhibition rate (%)=(average tumore weight of the control group−average tumor weight of the experiment group)/average tumor weight of the control group×100%

H22 ascites tumor inhibition experiment methods: Select well-growing H22 cells, dilute appropriately with sterile saline into a tumor cell suspension, cell counts for 10⁷·L⁻¹, each mouse intraperitoneal inoculation 0.2 ml (except for the normal control group). Treatment after 24 h of vaccination. Normal control group and negative control group were given saline 0.2 ml·each⁻¹·d⁻¹, intraperitoneal injection; positive control group were given cyclophosphamide 20 mg·kg⁻¹, 0.2 ml·each⁻¹d⁻¹, intraperitoneal injection. rLZ-8 treatment groups were given doses of tail venous injection respectively, 0.2 ml·each⁻¹·d⁻¹, in 10 consecutive days. Daily weighing, observe the weight changes of mice, drawing weight growth curves.

H22 subcutaneous tumor inhibition results: As can be seen from Table 7, all three rLZ-8 dose groups can inhibit the growth of S180, inhibition rates were 16.7%, 30.0%, 42.5%. There are very significant differences (p<0.01), rLZ-8 treatment group tumor weight compared with the negative control group.

TABLE 7
rLZ-8 effect on mouse transplanted inhibition of tumor
H22 ( x̅ ± s, n = 10)

| | Dosage | Tumor weight | Inhibition |
|---|---|---|---|
| Groups | (mg · kg⁻¹) | (g) | rate (%) |
| Negative control | — | 1.20 ± 0.02 | — |
| group | | | |
| CTX group | 20 | 0.45 ± 0.02* | 62.5 |
| rLZ-8 low-dose | 0.25 | 1.00 ± 0.03* | 16.7 |
| group | | | |
| rLZ-8 medium-dose | 0.5 | 0.84 ± 0.02* | 30.0 |
| group | | | |
| rLZ-8 high-dose | 1 | 0.69 ± 0.03* | 42.5 |
| group | | | |

Comparison with the negative group,
*p < 0.01

From Table 8 we know that the WBC counts of mice in these groups before medication were at the same level, compared with the negative control group there showed no differences (p>0.05). 10 days after treatment, the negative control group WBC counts were higher than that of the normal control group, rLZ-8 low-dose group and medium-dose group WBC counts and that of the negative control group showed no differences (p>0.05), the high-dose group compared with the normal control group showed no differences (p>0.05), the WBC counts in positive control group significantly decreased, compared with the normal control group and the negative control group were significantly different (p<0.01).

TABLE 8
rLZ-8 effect on mouse transplanted inhibition of tumor H22
interleukin ( x̅ ± s, n = 10)

| | | WBC numbers | |
|---|---|---|---|
| | dosage | Before | 10^{th} day of |
| Groups | (mg · kg⁻¹) | medication | medication |
| Normal control | — | 9.65 ± 0.28 | 9.69 ± 0.26 |
| group | | | |
| Negative control | — | 9.76 ± 0.31 | 10.49 ± 0.33 |
| group | | | |
| CTX group | 20 | 9.67 ± 0.43* | 4.49 ± 0.21* |
| rLZ-8 low-dose | 0.25 | 9.65 ± 0.33* | 10.53 ± 0.24** |
| group | | | |
| rLZ-8 medium-dose | 0.5 | 9.65 ± 0.38* | 10.43 ± 0.27** |
| group | | | |
| rLZ-8 hig-dose | 1 | 9.63 ± 0.41* | 9.86 ± 0.27** |
| group | | | |

Comparison with the negative group,
*p < 0.01;
**p > 0.05

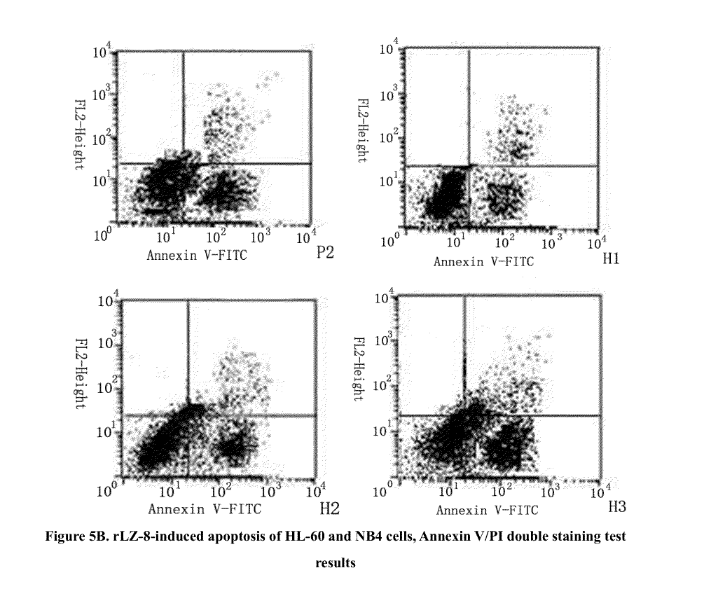

H22 ascites tumor inhibition experiment results: The results showed rLZ-8 groups of mice survive longer than the negative control group, in the negative control group mice showed loss of appetite, but there is a rapid growth in weight and less activity. It can be seen from FIG. 7 that rLZ-8 group, the growth weight of mice was bigger than the normal group on average, but less than the negative control group. It indicates that rLZ-8 inhibited the mouse peritoneal H22 tumor cells growth of mouse to a certain extent.

### Example 7

### rLZ-8 Fluorescent Labeling and Its Influence on Normal Tissue Cells and HI-60 Cells

### 1. rLz-8 Fluorescence of FITC Label

Fluorchrome (Fluorescein-5-Isothiocyanate, FITC), GL Biochem (Shanghai); Dimethyl sulfoxide; carbonate buffer solution (pH 8˜9.5) (Na₂CO₃ 4.3 g, NaHCO₃ 8.6 g add ddH₂O to 500 ml); Phosphorus buffered saline (PBS); Desalting Hiprep 26/10 desalting column; AKTA purifier; spectrophotometer as Hitachi model.

Mix the purified rLZ-8 (7.5 mg·ml⁻¹) 20 ml with carbonate buffer (pH 8.3), for dialysis overnight, weighing 3.75 mg FITC, add dimethyl sulfoxide (DMSO) 3.75 ml into FITC-DMSO solution. First put rLZ-8 in a small 50 ml beaker and then by mixing FITC-DMSO solution drops into rLZ-8 solution, and increase with PBS to 30 ml, stirring 4 h with magnetic stirrer at room temperature and lucifuging, desalting column with Desalting Hiprep 26/10 in AKTA purifier system to remove free fluorescein, 75 ml PBS elution, 280 nm, 495 nm detection, and peak collection.

Scan the prepared FITC-rLZ-8 (10-times diluted) at 220 nm˜520 nm, A495=0.445, A280=0.67, calculate tag efficiency (F/P) 3.80.

### 2. The Labeling Effect on Rat Myocardial Tissues

Leica CM1850 frozen slicer; wistar rat; fluorescence microscope 80i (Nikon); isotonic PBS buffer (pH 7.2); fetal bovine serum (FBS, Gibco); FITC-rLZ-8 is prepared by our laboratory. Cut the rats at neck and kill them, and strip the hearts, put them to frozen microtome till the temperature dropped to −20° C. for slicing, hatching for 1 h at 37° C. when the myocaridial and PBS preparation of FITC-rLZ-8 solution (100 ng·ml⁻¹), observing under fluorescence microscope to observe, and establish a blank control group.

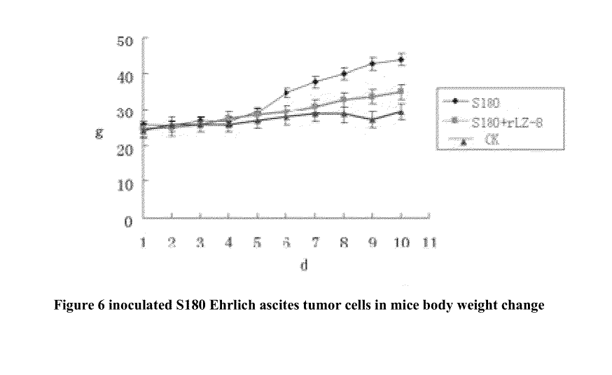

Under fluorescence microscope observation of myocardial tissues, it is without visible fluorescence, compared with the blank control group, there was no difference, see FIG. 8.

### 3. Labeling Effect on Primary Culture of Rabbit Chondrocytes

Japanese white rabbits (male, 2.5 kg) 4; surgical instruments; 0.25% trypsogen+0.02% EDTA; 0.2% collagenase ∥; D-Hanks; IMDM medium (50 ml·ml⁻¹, vitamin C, double-antibody); 0.025 mg·ml⁻¹ Poly-lysine solution; sterile water for injection (WFI).

Fix the experiment animals and execute them with air embolism, skinned abdominal in the middle and exposed the limbs, cutting the muscle fascia with scissors, disconnected the backbone from os longum, remove carefully the entire knee, hip and shoulder bones, with a rough pruning, immersed into D-Hanks. Moved the beaker with tissues into the super-clean units, move the tissues after pruning and cleansing into the 2nd sterile D-Hanks cup; assemble the knife, cutting a thin layer of cartilage off, use curved forceps move into the 6 cm culture incubator, washing with D-Hanks three times, discard majority of D-Hanks, cut the cartilage into 1 mm slices with ophthalmic scissors, discard majority of D-Hanks, spoon removing the broken bone vials into a 10 cm² culture flask, mix with trypsogen EDTA for digestion, 37° C., 30 min; replace the trypsogen for collagenase, put it into the hatching incubator of 37° C., taking out and shaking 5 min every 1 h, with 4-4.5 h, finish digestion. Prepare it to the cell suspension solution and mix 3 ml IMDM containing FBS 15%, 5×10⁴·ml⁻¹ vaccinated into the culture flask.

Vaccinated the cell on the 24-orifice plate, 0.5 ml/hole, establish a blank control group, hatching at 37° C. for 1 h with the final concentration of 0.25 μg·ml⁻¹ FITC-rLZ-8 0.5 ml., move the cells into 1.5 ml EP centrifugal (1000 r·min⁻¹, 7 min) washing with isotonic PBS three times, mix EP tube with 0.1 ml PBS, re-suspended the cells, check the suspension to make observation under the fluorescence microscope.

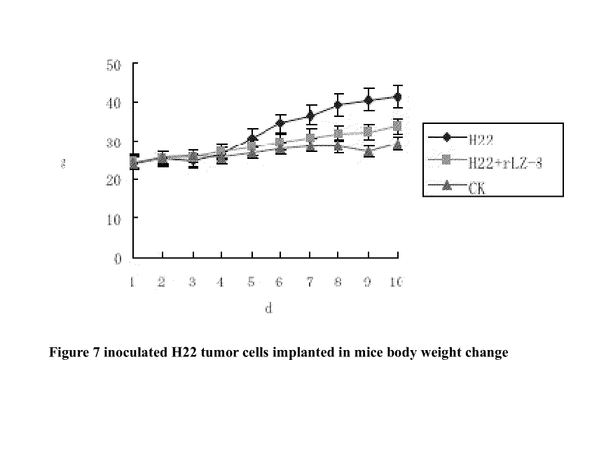

Observation under fluorescence microscope, the chondrocytes form of rabbits intact, without green fluorescence, compared with the control group there was no significant difference. Photos of experiment group are shown as in FIG. 9.

### 4. Labeling Effect of Hl-60 Cells

Fluorescence microscope 80i (Nikon), IMDM cell culture medium (Hyclone), fetal bovine serum (FBS, Gibco), FITC-rLZ-8 and isotonic PBS buffer (pH 7.2) Prepared by our laboratory.

Vaccinated the HL-60 by 2×106 inoculation on 24-hole plate, each hole 0.5 ml, with IMDM (2% FBS) culture medium prepared FITC-rLZ-8 for 100 ng·ml⁻¹, each hole 0.5 ml hatching (37° C.), establish a control group, experiment group 1 h and experiment group 6 h, draw the cells respectively from 1 h and 6 h, mixing into 1.5 ml EP tube, 1000 r·min⁻¹ centrifuge, clear the supernatant, wash with PBS 3 times, re-suspend after washing.

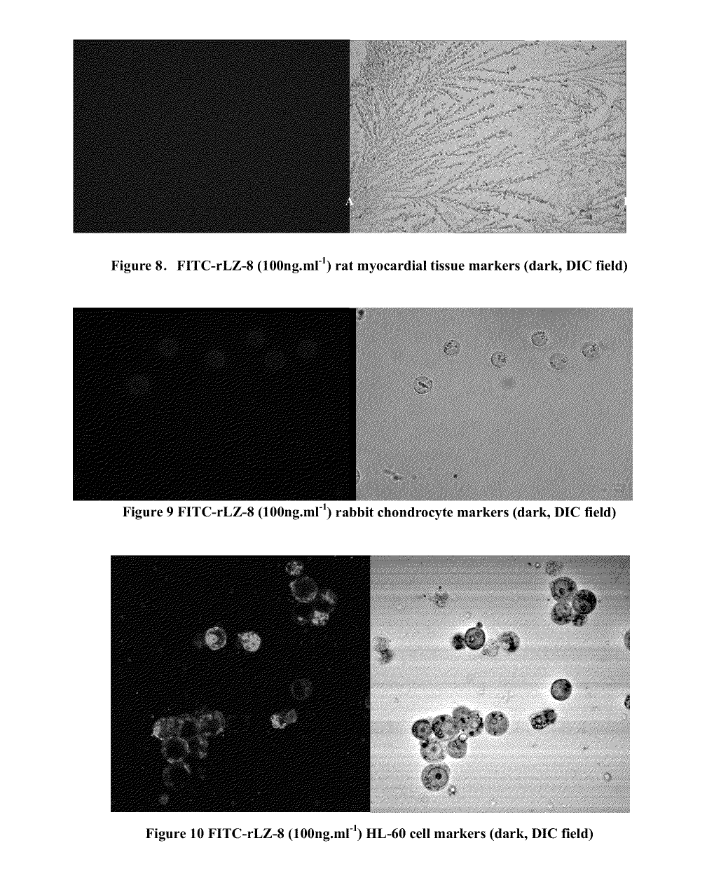

FIG. 10 observe under fluorescence microscope, with FITC-rLZ-8 incubation of 1 h and 6 h the HL-60 cells, with strong green fluorescence, and in group 6 h there was agglutination of cells, while in the blank control group, no green fluorescence, compared with former, there is a significant difference.

### Example 8

### rLZ-8 Effect in the Prevention of the Leucocyte in Mice

rLZ-8 preparation of sterile saline. Divide them into 5 μg·kg⁻¹, 2.5 μg·kg⁻¹, 1.25 μg·kg⁻¹, 0.62 μg·kg⁻¹ dose groups.

Genlei™ Scimax™ [Recombinant human granulocyte colony-stimulating factor injection (rhG-CSF)], production lot: 20060403; 75 μg/vial is prepared with sterile normal saline into the preparation of 3.2 μg·kg⁻¹, 0.2 ml/vial.

Cyclophosphamide (CP) for injection (the production lot number 050216; 200 mg/vial) is prepared with sterile normal saline into the preparation of 12.5 mg·kg⁻¹, 0.2 ml/vial.

Normal control group, several protein dose groups, positive control group (Genlei™ Scimax™).

Excluding the normal control group (given same normal saline), each group of mice were given tail vein injection of cyclophosphamide, 1.25 μg·kg⁻¹, 0.2 ml/vial, for 3 consecutive days. On 3rd day, rat tail venous blood sampling, sent to Jilin University First Hospital laboratory, cell analyze the number of WBC. After successful modeling, they were given to the above groups respective dose of rLZ-8 and positive drugs (Genlei™ Scimax™) treatment, the normal control group and CP groups were given same normal saline, on the 3rd , 7th and 14th day of the treatment to draw venous blood from mice tail respectively, sent to Jilin University First Hospital testing the numbers of WBC. Compare WBC changes before and after the treatment for analysis of drug efficacy.

As can be seen from Table 9, compared with the CP group, treatment on the 3rd day of rLZ-8 drug group in mice had significantly higher WBC, the difference is very significant, it recovers on the 7th day of treatment.

TABLE 9
rLZ-8 Effect on low-interleukin mice model ( x̅ ± s, n = 10)

| | Before | After | 3^{rd} day of | 7^{th} day of | 14^{th} day of |
|---|---|---|---|---|---|
| grouping | modeling | modeling | medication | medication | medication |
| Normal control | 10.5 ± 0.20 | 11.0 ± 0.25 | 10.8 ± 0.51 | 10.2 ± 0.11 | 11.2 ± 0.31 |
| group | | | | | |
| CP control group | 10.7 ± 0.56 | 1.5 ± 0.71 | 4.6 ± 0.34 | 8.3 ± 0.34 | 12.0 ± 0.32 |
| (20 μg · kg⁻¹) | | | | | |
| Genlei ™ Scimax ™ | 10.8 ± 0.22 | 1.5 ± 0.35 | 5.5 ± 0.12* | 22.7 ± 0.12* | 13.0 ± 0.19* |
| (3.2 μg · kg⁻¹) | | | | | |
| rLZ-8 (5 μg · kg⁻¹) | 9.92 ± 0.21 | 1.3 ± 0.32 | 5.0 ± 0.12* | 20.3 ± 0.11* | 14.3 ± 0.21* |
| rLZ-8 (2.5 μg · kg⁻¹) | 10.25 ± 0.39 | 1.5 ± 0.54 | 6.6 ± 0.77* | 22.8 ± 0.15* | 15.2 ± 0.11* |
| rLZ-8 (1.25 μg · kg⁻¹) | 10.4 ± 0.31 | 1.7 ± 0.45 | 8.1 ± 0.17* | 19.7 ± 0.17* | 13.9 ± 0.14* |
| rLZ-8 (0.62 μg · kg⁻¹) | 10.4 ± 0.91 | 1.4 ± 0.45 | 6.4 ± 0.41* | 27.2 ± 0.10* | 13.9 ± 0.17* |

Comparison between CP control group,
*p < 0.01

### Example 9

### rLZ-8 Effect in the Treatment of the Leucocyte in Rats

rLZ-8 preparation of sterile saline. Divide them into 20 μg·kg⁻¹, 10 μg·kg⁻¹, 5 μg·kg⁻¹, 2.5 μg·kg⁻¹, 1.25 μg·kg⁻¹, 0.625 μg·kg⁻¹, 0.31 μg·kg⁻¹ dose groups.

Genlei™ Scimax™ [Recombinant human granulocyte colony-stimulating factor injection (rhG-CSF)], production lot: 20060403; 75 μg/vial is prepared with sterile normal saline into the preparation of 9.45 μg·ml⁻¹, 0.1 ml/vial.

Cyclophosphamide (CP), the production lot number 050216; 200 mg /vial is prepared with sterile normal saline into the preparation of 20 mg·ml⁻¹, 0.1 ml/vial, namely, 49 mg·kg⁻¹.

Normal control group, protein low-dose group, protein medium-dose group, protein high-dose group, positive control group (Genlei™ Scimax™)

Excluding the normal control group (given same normal saline), each group of rats were given tail vein injection of cyclophosphamide, 20 mg·ml⁻¹, 0.1 ml/vial, for 3 consecutive days. On 3rd day, rat tail venous blood sampling, sent to Jilin University First Hospital laboratory, cell analyze the number of WBC. After successful modeling, they were given to the above groups respective dose of rLZ-8 and positive drugs (Genlei™ Scimax™) treatment, the normal control group and CP groups were given same normal saline, on the 3rd ,7th and 14th day of the treatment to draw venous blood from rats tail respectively, sent to Jilin University First Hospital testing the numbers of WBC. Compare WBC changes before and after the treatment for analysis of drug efficacy.

As can be seen from Table 10, compared with the CP group, treatment on the 3rd day of rLZ-8 drug group in rats had significantly higher WBC, the difference is very significant, it has returned to normal on the 7th day of treatment.

TABLE 10
rLZ-8 Effect on low-interleukin rat model ( x̅ ± s, n = 10)

| | Before | After | 3^{rd} day of | 7^{th} day of | 14^{th} day of |
|---|---|---|---|---|---|
| grouping | modeling | modeling | medication | medication | medication |
| Normal control | 11.8 ± 0.21 | 11.2 ± 0.41 | 10.5 ± 0.29 | 11.0 ± 0.85 | 12.0 ± 0.11 |
| group | | | | | |
| CP control group | 11.56 ± 0.89 | 0.8 ± 0.71 | 0.6 ± 0.32 | 11.2 ± 0.12 | 11.5 ± 0.12 |
| (20 μg · kg⁻¹) | | | | | |
| Genlei ™ Scimax ™ | 11.7 ± 0.14 | 0.6 ± 0.23 | 1.4 ± 0.11* | 13.1 ± 0.21* | 10.3 ± 0.14 |
| (9.45 μg · kg⁻¹) | | | | | |
| rLZ-8 (20 μg · kg⁻¹) | 11.2 ± 0.11 | 0.7 ± 0.34 | 1.6 ± 0.33* | 15.8 ± 0.13* | 9.8 ± 0.19* |
| rLZ-8 (10 μg · kg⁻¹) | 11.1 ± 0.34 | 0.6 ± 0.56 | 1.6 ± 0.71* | 15.8 ± 0.33* | 10.9 ± 0.21* |
| rLZ-8 (5 μg · kg⁻¹) | 11.4 ± 0.22 | 0.8 ± 0.79 | 2.6 ± 0.64* | 17.6 ± 0.23* | 11.2 ± 0.26 |
| rLZ-8 (2.5 μg · kg⁻¹) | 11.0 ± 0.98 | 0.8 ± 0.12 | 1.7 ± 0.18* | 12.8 ± 0.11* | 10.6 ± 0.29* |
| rLZ-8 (1.25 μg · kg⁻¹) | 12.0 ± 0.24 | 0.7 ± 0.11 | 2.8 ± 0.12* | 11.0 ± 0.47* | 10.3 ± 0.45* |
| rLZ-8 (0.62 μg · kg⁻¹) | 11.7 ± 0.45 | 0.8 ± 0.74 | 5.2 ± 0.14* | 12.8 ± 0.74* | 12.1 ± 0.24* |
| rLZ-8 (0.31 μg · kg⁻¹) | 14.2 ± 0.11 | 0.9 ± 0.12 | 1.9 ± 0.17* | 13.5 ± 0.13* | 11.0 ± 0.31 |

Comparison between CP control group,
*p < 0.01

### Example 10

### rLZ-8 Effect in the Prevention of Model of Mice by Radiation

rLZ-8 preparation of sterile saline. Divide them into 5 μg·kg⁻¹, 2.5 μg·kg⁻¹, 1.25 μg·kg⁻¹, 0.62 μg·kg⁻¹ dose groups. 0.2 ml/vial.

Genlei™ Scimax™ [Recombinant human granulocyte colony-stimulating factor injection (rhG-CSF)], production lot: 20060403; 150 μg/vial is prepared with sterile normal saline into the preparation of 3.2 μg·kg⁻¹, 0.2 ml/vial.

The mice were irradiated by 7.5 Gy γ-ray (180 mV, 15 mA) and then divided into seven groups (n=10): (A) negative control; (B) 5 μg·kg-1 dose; (C) 2.5 μg·kg-1 dose; (D) 1.25 μg·kg-1 dose; (E) 0.62 μg·kg-1 dose; (F) positive control (Genlei™ Scimax™); (G) model group. Before modeling 5 days, mice in each group were administered with Genlei™ Scimax™ and rLZ-8 for 5 days, on the 5^{th} day, mice were radiated, for 5 days, and on the 7^{th} day, drew blood from tail, tested the number of WBC, weight spleen, calculated index of spleen.

As is shown in table 12, the amount of WBC in each rLZ-8's dose group has a significant difference with model group. (p<0.05) As is shown in Table 11, rLZ-8 has stimulated spleen growth, spleen index and colony forming unit-spleen of each rLZ-8's dose group is higher than that of model group.

TABLE 11
The results of effect in the prevention of model of mice by
radiation ( x̅ ± s, n = 10)

| | Before | 5^{th} day | | |
|---|---|---|---|---|
| Group | medicine | (rLZ-8) | 5^{th} day | 7^{th} day |
| Normal control | 11.5 ± 0.20 | 11.0 ± 0.25 | 10.8 ± 0.21 | 9.8 ± 0.11 |
| Model control | 11.5 ± 0.56 | 14.4 ± 0.71 | 0.30 ± 0.33 | 0.26 ± 0.33 |
| Genlei ™ Scimax ™ | 11.4 ± 0.22 | 21.8 ± 0.35* | 0.26 ± 0.20 | 0.22 ± 0.12 |
| 3.2 μg · } | | | | |
| rLZ-8 (5 μg · kg⁻¹) | 10.3 ± 0.21 | 19.5 ± 0.32* | 0.32 ± 0.18 | 0.53 ± 0.35* |
| rLZ-8 (2.5 μg · kg⁻¹) | 11.7 ± 0.39 | 12.8 ± 0.54* | 0.32 ± 0.15 | 0.45 ± 0.20* |
| rLZ-8 (1.25 μg · kg⁻¹) | 10.4 ± 0.31 | 12.5 ± 0.45* | 0.25 ± 0.11 | 0.46 ± 0.11* |
| rLZ-8 (0.62 μg · kg⁻¹) | 11.2 ± 0.91 | 10.7 ± 0.45* | 0.16 ± 0.31 | 0.40 ± 0.12* |

Comparison between model group,
*p < 0.05

TABLE 12
The results of effect in the treatment of model of mice by radiation ( x̅ ± s, n = 10)

| | Before | | | |
|---|---|---|---|---|
| Group | medicine | 5^{th} day | 7^{th} day | 9^{th} day |
| Normal control | 10.5 ± 0.20 | 11.0 ± 0.25 | 10.0 ± 0.25 | 12.8 ± 0.51 |
| Model control | 11.0 ± 0.56 | 0.7 ± 0.71 | 0.2 ± 0.71 | 0.61 ± 0.01 |
| Genlei ™ Scimax ™ | 11.9 ± 0.22 | 0.68 ± 0.35 | 0.58 ± 0.35* | 0.80 ± 0.11* |
| (3.2 μg · kg⁻¹) | | | | |
| rLz-8 (5 μg · kg⁻¹) | 11.2 ± 0.21 | 0.84 ± 0.32* | 0.31 ± 0.32* | 0.90 ± 0.14* |
| rLz-8 (2.50 μg · kg⁻¹) | 12.9 ± 0.39 | 0.69 ± 0.54 | 0.56 ± 0.54* | 0.54 ± 0.13* |
| rLz-8 (1.25 μg · kg⁻¹) | 9.6 ± 0.31 | 0.82 ± 0.45* | 0.65 ± 0.45* | 0.70 ± 0.12* |
| rLz-8 (0.62 μg · kg⁻¹) | 10.4 ± 0.91 | 0.73 ± 0.43* | 0.26 ± 0.43* | 0.64 ± 0.01 |

Comparison between model group,
*p < 0.05

### Example 11

### rLZ-8 Effect in the Treatment of Model of Mice by Radiation

rLZ-8 preparation of sterile saline. Divide them into 5 μg·kg⁻¹, 2.5 μg·kg⁻¹, 1.25 μg·kg⁻¹, 0.62 μg·kg⁻¹ dose groups. 0.2 ml/vial.

Genlei™ Scimax™ [Recombinant human granulocyte colony-stimulating factor injection (rhG-CSF)], production lot: 20060403; 150 μg/vial is prepared with sterile normal saline into the preparation of 3.2 μg·kg⁻¹, 0.2 ml/vial.

The mice were irradiated by 7.5 Gy γ-ray (180 mV, 15 mA) and then divided into seven groups (n=10): (A) negative control; (B) 5 μg·kg-1 dose; (C) 2.5 μg·kg-1 dose; (D) 1.25 μg·kg-1 dose; (E) 0.62 μg·kg-1 dose; (F) positive control (Genlei™ Scimax™); (G) model group. Together with modeling, mice in each group were administered with Genlei™ Scimax™ and rLZ-8 for 9 days, drew blood from tail, tested the number of WBC, weight spleen, calculated index of spleen.

As is shown in table 12, the amount of WBC in each rLZ-8's dose group has a significant difference with model group. (p<0.05)As is shown in table 13, rLZ-8 has stimulated spleen growth, spleen index and colony forming unit-spleen of each rLZ-8's dose group was higher than that of model group. (p<0.05)

TABLE 13
The number of colony forming unit-spleen in model of mice by
radiation ( x̅ ± s, n = 10)

| Group | number | index | growth rate |
|---|---|---|---|
| Normal control | 4.4 ± 0.10 | 0.17 ± 0.11 | |
| Genlei ™ Scimax ™ | 6.0 ± 0.11* | 0.20 ± 0.10* | 26% |
| (3.2 μg · kg⁻¹) | | | |
| rLz-8 (5 μg · kg⁻¹) | 9.7 ± 0.16* | 0.22 ± 0.09 | 54% |
| rLz-8 (2.50 μg · kg⁻¹) | 14.3 ± 0.03* | 0.52 ± 0.12* | 69% |
| rLz-8 (1.25 μg · kg⁻¹) | 6.3 ± 0.21* | 0.24 ± 0.13 | 30% |
| rLz-8 (0.62 μg · kg⁻¹) | 8.9 ± 0.41* | 0.50 ± 0.10* | 50% |

Comparison between model group,
*p < 0.01

### Example 12

### rLZ-8 Inhibiting Systemic Allergic Reaction in Mice

Prepare rLZ-8 700 μg/ml⁻¹ with saline and Aluminium hydroxide with PBS buffer. Male mice, 15 vial, were divided into 4 groups: two rLZ-8 dosage groups, positive group, normal control group. Administration strategies were as follows: in rLZ-8 dosage groups, injected mice with 0.1 ml/10 g, twice a week, for 3 weeks, after starting injection first week, mice were give mixture of 1 mg BSA and 0.2 ml hydroxide, after sensitization 17th day, injected with 1 mg BSA 0.2 ml, observed reactions of mice; in normal control group, injected with saline, same volume. Positive reaction standard: tic or death. Negative reaction standard: normal.

As is shown in table 14, rLZ-8 could inhibit systemic allergic reaction caused by BSA in mice.

TABLE 14
The results of rLZ-8 inhibiting systemic allergic reaction in mice

| | | | | results | |
|---|---|---|---|---|---|
| | | | rLZ-8 | Sensitization | Death |
| group | sensitization | shock | treatment | number/total | number/total |
| Normal | BSA (i.p.) | BSA (i.v.) | − | 10/10 | 1/10 |
| control | | | | | |
| Positive | BSA (i.p.) | OA (i.v.) | − | 0/10 | 0/10 |
| control | | | | | |
| rLZ-8dose1 | BSA (i.p.) | BSA (i.v.) | + | 5/10 | 0/10 |
| rLZ-8dose 2 | BSA (i.p.) | BSA-rLZ-8 | + | 9/10 | 0/10 |
| | | (i.v.) | | | |

### Example 13

### rLZ-8 Hemolysis Test

rLZ-8 prepares 5% of glucose solution into the preparation of 1 mg·ml⁻¹; blood cell suspension preparation: human blood 4 ml, 1000 r/min centrifugal 10 min, clears supernatant. By adding 5% glucose solution about 10 times of the volume to erythrocyte sedimentation, shaking, centrifugal 1000 r/min 20 min, clear the supernatant, repeat the washing 2-3 times until the supernatant is not significantly red. Prepare the obtained erythrocyte with 5% glucose solution into 2% suspension for experiment.

Get 28 clean test tubes and number them, No. 1-5 for rLZ-8 drug group, No. 6 for negative control group (5% glucose solution), No. 7 for positive control tube (distilled water), and a total of 4 parallel comparison tubes. By supplying 2% erythrocyte suspension, 5% glucose or distilled water in turn, shaking, put them into incubators 37° C.±0.5° C. immediately for incubation. Observe them every 15 min, 1 h after observe them once for 3 hours. Finishing the observation, put all the solution from the tubes into centrifugal drying tubes, 1500 r, 25 min. Clear the supernatant, read the blank distilled water tube for OD value to calculate the rate of hemolysis.

We can see in Table 15 that rLZ-8 hemolytic rate of drug group from 1-5 group <5%, there is no sign of hemolytic reaction.

TABLE 15
Experiment results of rLZ-8 hemolysis on human erythrocyte solubilization ( x̅ ± s, n = 10)

| Serial number of test tubes | 1 | 2 | 3 | 4 | 5 |
|---|---|---|---|---|---|
| Hemolysis rate (%) | 0.70 ± 0.03 | 0.74 ± 0.04 | 0.63 ± 0.04 | 0.65 ± 0.04 | 0.59 ± 0.07 |

### Example 14

### Influence of rLZ-8 on Rat Myelogram

Waster rats 9, about 100 g. Prepare rLZ-8 with sterile saline solution for three dose groups of 60 mg·kg⁻¹, 30 mg·kg⁻¹, 15 mg·kg⁻¹.

Normal control group 3 (rats), protein low-dose group 2, protein medium-dose group 2, and protein high-dose group 2. rLZ-8 drug group rats, are given different doses of rLZ-8 tail vein injection respectively, 1 time/day; the normal control group are given equivalent saline. On the 7th day of injection, check the right side of the thigh bone marrow for smears.

Compare the rat bone marrow smear with the normal control group, no abnormal appears.

### Example 15

### rLZ-8 on Human Red Blood Cell Cohesion Effects

A, B, O and AB blood type, from the healthy volunteers 2 ml respectively, SRBC (sheep red cell) 2 ml. Centrifuge the above RBC 1200 g·min⁻¹ for 10 min, clears the supernatant and wash with 5 ml PBS, repeat the above operation 3-5 times, and then uses 0.01 mol/L PBS suspension preparation of 1.5%. Prepare rLZ-8 with normal saline to final concentrations of 50 μg·ml⁻¹, 25 μg·ml⁻¹, 12.5 μg·ml⁻¹, 6.25 μg·ml⁻¹, 3.13 μg·ml⁻¹, 1.56 μg·ml⁻¹, 0.78 μg·ml⁻¹, 0.39 μg·ml⁻¹, 0.20 μg·ml⁻¹, 0.10 μg·ml⁻¹, 0.05 μg·ml⁻¹, 0.03 μg·ml⁻¹. Plant agglutinin (PHA) preparation Ibid.

Divide experiment subjects into rLZ-8 concentration groups, positive drug control, and normal control group.

On 96-orifice Hemagglutination board, we add A-type 1.5%, erythrocyte 25 μl/hole, and then add 0.2% gelatin, 75 μl/hole. Drug groups are added different concentrations of rLZ-8, till the final concentration as above-noted; positive drug control group PHA 25 μg/hole; normal control group PBS 25 μl/hole. 6 parallel control groups in each group. Shaking for 30 s under normal room temperature, 37° C. incubation, observe the subjects 1 h later. For B-type, AB-type, O-type, the same experiment methods are as SRBC (sheep red cell) above.

Table 16 below indicates that positive drug PHA is agglutinative on all four types of human red blood cells and sheep red blood cells; rLZ-8 is not agglutinative on four types of human red blood cells, while at 12.5 μg·ml⁻¹-50 μg·ml⁻¹ concentration SRBC shows active agglutination.

able 16 rLZ-8 Experiment result of 4 types of human red blood cell agglutination

| | Normal | rLZ-8 medicine group (μg · ml⁻¹) | | | | | | | | | | | |
|---|---|---|---|---|---|---|---|---|---|---|---|---|---|
| grouping | group | 50 | 25 | 12.5 | 6.25 | 3.13 | 1.56 | 0.78 | 0.39 | 0.20 | 0.10 | 0.05 | 0.03 |
| A | — | — | — | — | — | — | — | — | — | — | — | — | — |
| B | — | — | — | — | — | — | — | — | — | — | — | — | — |
| O | — | — | — | — | — | — | — | — | — | — | — | — | — |
| AB | — | — | — | — | — | — | — | — | — | — | — | — | — |
| SRBC | — | +++ | ++ | + | — | — | — | — | — | — | — | — | — |
| (sheep red cell) | | | | | | | | | | | | | |

### Example 16

### The Recombinant Ganoderma Lucidum Immune Regulatory Protein Anti-Tumor Preparation

Through above pharmacological experiments, it proves that the effect of Recombinant Ganoderma Lucidum Immune Regulatory Protein rLZ-8 anti-tumor and raising level of interleukin is very significant, and non-toxic without side-effect. Therefore, it can be said that the Recombinant Ganoderma Lucidum Immune Regulatory Protein rLZ-8 as an anti-tumor preparation is suitable for drug use and is safe.

This invention of Recombinant Ganoderma Lucidum Immune Regulatory Protein rLZ-8 as an application of anti-cancer drug can be given orally and non-intestinal drug delivery. Dosage can be decided by symptoms, age, weight and other factors. For adults, the oral dosage for per person is 10-1000 mg, several times a day; non-intestinal delivery of 10-100 mg, several times a day.

In this invention, we have kits of oral tablets, pills and capsules (including the hard and soft capsules), these preparation formulations include the Recombinant Ganoderma Lucidum Immune Regulatory Protein rLZ-8 and at least one inert diluent (such as lactose, mannose alcohol, glucose, starch, poly vinyl pyrrolidone), it can also be joined by acceptable additives such as lubricants, disintegrants, stabilizers other than the inert diluent pharmacology. If it is necessary, tablets or pills can be coated by gastric soluble or enteric coating material on one or more than one layer of coatings. Non-intestinal injection includes Recombinant Ganoderma Lucidum Immune Regulatory Protein rLZ-8 and at least one inert water diluent (such as distilled injection water and normal saline), Recombinant Ganoderma Lucidum Immune Regulatory Protein rLZ-8 can also be made freeze-dried powder; we may dissolve the powder in inert diluent water for injection before use.

### (1) Preparation Case 1

Get Recombinant Ganoderma Lucidum Immune Regulatory Protein rLZ-8 1000 mg; dissolve in 100 ml of sterile saline, mixing evenly, then sub-pack them into rLZ-8 10 mg/ml/vial concentration solution injection bottle; sealed, sterilized and made to be products. Other items shall conform to the pharmacopoeia of the People's Republic of China 2005 edition under the requirements of injection.

### (2) Preparation Case 2

Get Recombinant Ganoderma Lucidum Immune Regulatory Protein rLZ-8 100 g; medicinal starch 0.5 kg, in accordance with the publicly-known technology and equipment manufacture them into capsule forms, rlz8 10 mg/tablets and other items that shall conform to the pharmacopoeia of the People's Republic of China 2005 edition under the requirements of capsules.

### (3) Preparation Case 3

Get Recombinant Ganoderma Lucidum Immune Regulatory Protein rLZ-8 100 g; microcrystalline cellulose 560 g; anhydrous lactose 380 g; magnesium stearate 200 g, in accordance with the publicly-known technology and equipment manufacture them into pills/tablets, rLZ-8 10 mg/tablet, other itmes shall conform to the pharmacopoeia of the the People's Republic of China 2005 edition under the requirements of tablets.

### (4) Preparation Case 4

Get Recombinant Ganoderma Lucidum Immune Regulatory Protein rLZ-8 moderate, items shall conform to the pharmacopoeia of the People's Republic of China 2005 edition under the requirements of oral liquid, manufacture oral liquid in accordance with the publicly-known technology and equipment.

1-17. (canceled)

18. A method of treating a tumor, comprising: administering to a subject, an effective amount of a recombinant Ganoderma Lucidum Immunoregulatory Protein (rLZ-8) having a particular spatial structure and being encoded by SEQ ID: NO 1, wherein the tumor includes at least one selected from the group consisting of leukemia, liver cancer and ascites cancer.

19. The method of claim 18, wherein the amino acid sequence of LZ-8 is SEQ ID: NO 2,
wherein a crystal structure of LZ-8 solved at 2.10 Å resolution belongs to the space group of P212121, with a unit cell of a=33.19 Å, b=86.99 Å, c=92.13 Å and α=β=γ=90°, and wherein an overall fold of LZ-8 consists of an N-terminal dimerization domain and a C-terminal FNIII domain, the N-terminal domain being composed of an α-helix and a β-strand that sustains the dimerization via domain swapping, forming a dumb-bell-shaped dimer, the C-terminal FNIII domain belonging to the immunoglobulin-like beta-sandwich fold and comprising a sandwich structure of two β-sheets (I and II) formed by β-strands A-B-E and G-F-C-D, respectively, the amino acid sequences of the β-strands A-B-E and G-F-C-D being the following: A. 21-TPNWGRG-27 (SEQ ID: NO 3); B. 34-IDTVTFP-39 (SEQ ID: NO 4); C. 48-YTYRVAV-54 (SEQ ID: NO 5); D. 57-RNLGVK P-63 (SEQ ID: NO 6); E. 72-SQKVN-76 (SEQ ID: NO 7); F. 91-TIQVFVVDPD-100 (SEQ ID: NO 8); G. 102-NNDFIIAQW-110 (SEQ ID: NO 9).

20. The method of claim 18, wherein the amount of the rLZ-8 is between 0.25 to 20 μg·kg⁻¹.
